(19) United States
(12) Patent Application Publication
(10) Pub. No.: US 2015/0376081 A1
(43) Pub. Date: Dec. 31, 2015

(54) METHODS AND APPARATUSES FOR PRODUCING LINEAR ALKYLBENZENE FROM RENEWABLE OILS

(71) Applicant: UOP LLC, Des Plaines, IL (US)

(72) Inventors: Debarshi Majumder, Forest Park, IL (US); Geoffrey William Fichtl, Chicago, IL (US)

(21) Appl. No.: 14/320,088

(22) Filed: Jun. 30, 2014

(51) Int. Cl. C07C 1/22; C07C 4/02; C07C 5/22; C07C 2/42; C07C 2/74; C07C 5/327

(52) U.S. Cl. CPC C07C 1/22; C07C 2/74; C07C 5/327; C07C 5/22; C07C 2/42; C07C 4/02

(57) ABSTRACT

Apparatuses and methods for producing linear alkylbenzene products from renewable oils are provided. An exemplary method includes deoxygenating the renewable oil in a deoxygenation zone to form paraffins. The method fractionates the paraffins to form first and second fractions. The method processes the first fraction of paraffins in a reforming zone to form a reformate stream and recovers a first hydrogen stream from the reforming zone. The method includes forming a LAB stream in a LAB production zone from the second fraction of paraffins and a portion of the reformate stream. Further, the method includes recovering a second hydrogen stream in the LAB production zone and recycling the first hydrogen stream from the reforming zone and the second hydrogen stream from the LAB production zone, wherein substantially all hydrogen recovered from the reforming zone and the LAB production zone is recycled.

### TECHNICAL FIELD

The present disclosure relates generally to apparatuses and methods for producing linear alkylbenzene from renewable oils, and more particularly relates to apparatuses and methods for producing alkylbenzene from renewable oils using recycled hydrogen.

### BACKGROUND

Linear alkylbenzenes are organic compounds with the formula C₆H₅CₙH₂ₙ₊₁. While n can have any practical value, current commercial use of alkylbenzenes requires that n lie from 10 to 16, or more specifically from 10 to 13, from 12 to 15, or from 12 to 13. These specific ranges are often required when the alkylbenzenes are used as intermediates in the production of surfactants for detergents. Because the surfactants created from alkylbenzenes are biodegradable, the production of alkylbenzenes has grown rapidly since their initial uses in detergent production in the 1960s.

While detergents made utilizing alkylbenzene-based surfactants are biodegradable, typical processes for creating linear alkylbenzenes are not based on renewable sources. Specifically, linear alkylbenzenes are currently produced from kerosene extracted from the earth. Due to the growing environmental concerns over fossil fuel extraction and economic concerns over exhausting fossil fuel deposits, there may be support for using an alternate source for biodegradable surfactants in detergents and in other industries.

Accordingly, it is desirable to provide apparatuses and methods for producing linear alkylbenzene from renewable oils, i.e., oils that are not extracted from the earth. Further, it is desirable to provide apparatuses and methods that produce linear alkylbenzenes from renewable oils while recycling all hydrogen streams generated during the process. Furthermore, other desirable features and characteristics will become apparent from the subsequent detailed description and the appended claims, when taken in conjunction with the accompanying drawing and this background.

### BRIEF SUMMARY

Apparatuses and methods for producing linear alkylbenzene products from natural oils are provided. In an exemplary embodiment, a method for producing a linear alkylbenzene product from a renewable oil includes deoxygenating the renewable oil in a deoxygenation zone to form a paraffin stream. The method fractionates the paraffin stream to form first and second fractions of paraffins. The method processes the first fraction of paraffins in a reforming zone to form a reformate stream and recovers a first hydrogen stream from the reforming zone. The method includes forming a linear alkylbenzene stream in a linear alkylbenzene production zone from the second fraction of paraffins and a portion of the reformate stream. Further, the method includes recovering a second hydrogen stream in the linear alkylbenzene production zone and recycling the first hydrogen stream from the reforming zone and the second hydrogen stream from the linear alkylbenzene production zone, wherein substantially all hydrogen recovered from the reforming zone and the linear alkylbenzene production zone is recycled.

In another exemplary embodiment, a method for producing a linear alkylbenzene product from a renewable oil includes deoxygenating the renewable oil to form a paraffin stream. The method fractionates the paraffin stream to form an overhead fraction of paraffins, a sidedraw fraction of paraffins, and a bottom fraction of paraffins. The method includes isomerizing and/or cracking the bottom fraction of paraffins to form an isomerized/cracked stream and reforming the overhead fraction of paraffins and the isomerized/cracked stream to form a reformate stream and a first hydrogen stream. Further, the method includes reacting the sidedraw fraction of paraffins with a portion of the reformate stream to form a linear alkylbenzene stream and a second hydrogen stream. The renewable oil is deoxygenated in the presence of hydrogen from the first hydrogen stream and the second hydrogen stream to form the paraffin stream.

In accordance with another embodiment, an apparatus for producing a linear alkylbenzene product from a renewable oil is provided. The apparatus includes a deoxygenation zone for deoxygenating the renewable oil to form a paraffin stream. The apparatus also includes a fractionation unit in fluid communication with the deoxygenation zone to receive and fractionate the paraffin stream into a first fraction of paraffins, a second fraction of paraffins, and a third fraction of paraffins. A reforming zone is provided in fluid communication with the fractionation unit to receive the first fraction of paraffins and to form a reformate stream and a first hydrogen stream. The reforming zone recycles the first hydrogen stream within the apparatus. Further, the apparatus includes a linear alkylbenzene production zone in fluid communication with the fractionation unit and with the reforming zone to receive the second fraction of paraffins and a portion of the reformate stream to form a linear alkylbenzene stream and second hydrogen stream. The linear alkylbenzene production zone recycles the second hydrogen stream within the apparatus.

### BRIEF DESCRIPTION OF THE DRAWING

Embodiments of the apparatuses and methods for producing linear alkylbenzene from renewable oils will hereinafter be described in conjunction with the following drawing figure wherein:

FIG. 1 schematically illustrates an apparatus for producing linear alkylbenzene from renewable oils in accordance with an exemplary embodiment.

### DETAILED DESCRIPTION

The following Detailed Description is merely exemplary in nature and is not intended to limit the apparatuses and methods for producing linear alkylbenzene from renewable oils claimed herein. Furthermore, there is no intention to be bound by any theory presented in the preceding Background or the following Detailed Description.

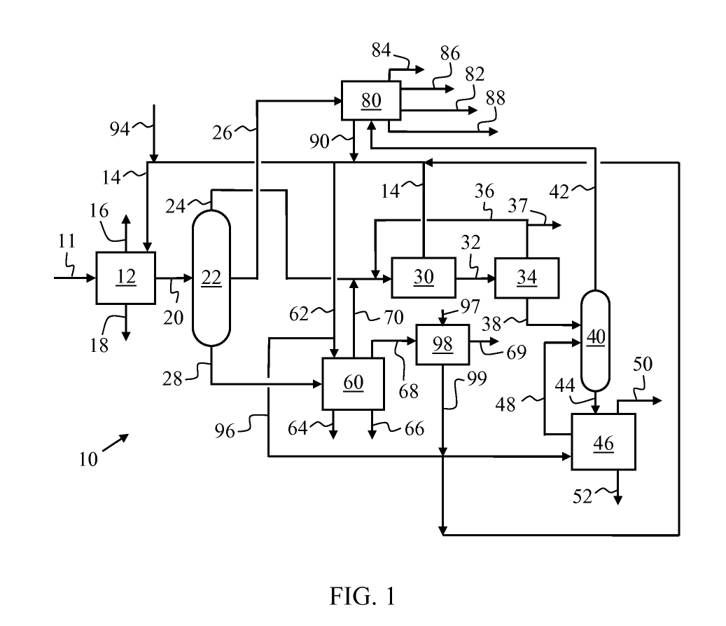

Various embodiments contemplated herein relate to methods and apparatuses for producing a linear alkylbenzene product from renewable oils. In FIG. 1, an exemplary apparatus 10 for producing a linear alkylbenzene product from a renewable oil 11 is illustrated. As used herein, renewable oils are those derived from biomass such as plant or algae matter. Renewable oils are not derived from petroleum or other fossil fuels. In certain embodiments, the renewable oils include one or more of coconut oil, palm kernel oil, babassu oil, castor oil, cooking oil, and other vegetable, nut or seed oils. The renewable oils typically comprise triglycerides, free fatty acids, or a combination of triglycerides and free fatty acids.

As used in the present disclosure, the term “renewable” denotes that the carbon content of the renewable hydrocarbon (paraffins, olefins, aromatics, alkylbenzene, linear alkylbenzene or subsequent products prepared from renewable hydrocarbons), is from a “new carbon” source as measured by ASTM test method D6866-05, “Determining the Bio-based Content of Natural Range Materials Using Radiocarbon and Isotope Ratio Mass Spectrometry Analysis”, hereby incorporated by reference in its entirety. This test method measures the ¹⁴C/¹²C isotope ratio in a sample and compares it to the ¹⁴C/¹²C isotope ratio a standard 100 mass % bio-based material to give percent bio-based content of the sample. Additionally, “Bio-based materials” are organic materials in which the carbon comes from recently, on a human time scale, fixated CO₂ present in the atmosphere using sunlight energy, photosynthesis. On land, this CO₂ is captured or fixated by plant life such as agricultural crops or forestry materials. In the oceans, the CO₂ is captured or fixated by photosynthesizing bacteria or phytoplankton. For example, a bio-based material has a ¹⁴C/¹²C isotope ratio greater than 0. Contrarily, a fossil-based material has a ¹⁴C/¹²C isotope ratio of about 0. The term “renewable” with regard to compounds such as hydrocarbons (paraffins, olefins, di-olefins, aromatics, alkylbenzene, linear alkylbenzene etc.) also refers to compounds prepared from biomass using thermochemical methods such as (e. g., Fischer-Tropsch catalysts), biocatalysts (e. g., fermentation), or other processes, for example.

A small amount of the carbon atoms in the atmospheric carbon dioxide is the radioactive isotope ¹⁴C. This ¹⁴C carbon dioxide is created when atmospheric nitrogen is struck by a cosmic ray generated neutron, causing the nitrogen to lose a proton and form carbon of atomic mass 14 (¹⁴C), which is then immediately oxidized, to carbon dioxide. A small but measurable fraction of atmospheric carbon is present in the form of ¹⁴C. Atmospheric carbon dioxide is processed by green plants to make organic molecules during the process known as photosynthesis. Virtually all forms of life on Earth depend on this green plant production of organic molecules to produce the chemical energy that facilitates growth and reproduction. Therefore, the ¹⁴C that forms in the atmosphere eventually becomes part of all life forms and their biological products, enriching biomass and organisms which feed on biomass with ¹⁴C. In contrast, carbon from fossil fuels does not have the signature ¹⁴C/¹²C ratio of renewable organic molecules derived from atmospheric carbon dioxide. Furthermore, renewable organic molecules that biodegrade to CO₂ do not contribute to an increase in atmospheric greenhouse gases as there is no net increase of carbon emitted to the atmosphere.

Assessment of the renewably based carbon content of a material can be performed through standard test methods such as using radiocarbon and isotope ratio mass spectrometry analysis. ASTM International (formally known as the American Society for Testing and Materials) has established a standard method for assessing the bio-based content of materials. The ASTM method is designated ASTM-D6866.

The application of ASTM-D6866 to derive “biobased content” is built on the same concepts as radiocarbon dating, but without use of the age equations. The analysis is performed by deriving a ratio of the amount of radiocarbon (¹⁴C) in an unknown sample compared to that of a modern reference standard. This ratio is reported as a percentage with the units “pMC” (percent modem carbon). If the material being analyzed is a mixture of present day radiocarbon and fossil carbon, which contains very low levels of radiocarbon, then the pMC value obtained correlates directly to the amount of biomass material present in the sample.

In the illustrated embodiment, the renewable oil 11 is deoxygenated in a deoxygenation zone 12 that also receives a hydrogen stream 14. As used herein, the term “zone” can refer to an area including one or more equipment items and/or one or more sub-zones. Equipment items can include one or more reactors or reactor vessels, heaters, exchangers, pipes, pumps, compressors, and controllers. Additionally, an equipment item, such as a reactor, dryer, or vessel, can further include one or more zones or sub-zones.

In the deoxygenation zone 12, the triglycerides and fatty acids in the renewable oil 11 are deoxygenated and converted into normal paraffins. Structurally, triglycerides are formed by three, typically different, fatty acid molecules that are bonded together with a glycerol bridge. The glycerol molecule includes three hydroxyl groups (HO—) and each fatty acid molecule has a carboxyl group (COOH). In triglycerides, the hydroxyl groups of the glycerol join the carboxyl groups of the fatty acids to form ester bonds. Therefore, during deoxygenation, the fatty acids are freed from the triglyceride structure and are converted into normal paraffins. The glycerol is converted into propane, and the oxygen in the hydroxyl and carboxyl groups is converted into either water or carbon dioxide. The deoxygenation reaction for fatty acids and triglycerides are respectively illustrated as:

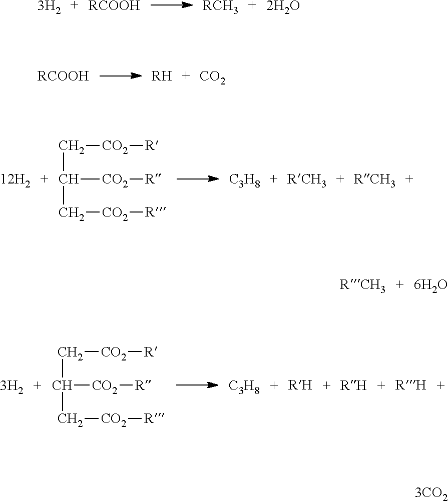

During the deoxygenation reaction, the length of a paraffin chain Rⁿ created will vary by a value of one depending on the exact reaction pathway. For instance, if carbon dioxide or carbon monoxide is formed, then the chain will have one fewer carbon than the fatty acid source. If water is formed, then the chain will match the length of the chain in the fatty acid source. FIG. 1, a stream 16 including light paraffins, such as propane and paraffins having a lower boiling temperature than propane, hydrogen sulfide, carbon monoxide and carbon dioxide and a stream 18 including water are formed and exit the deoxygenation zone 12.

In FIG. 1, a deoxygenated stream 20 containing normal paraffins, such as C₄-C₃₂ paraffins, exits the deoxygenation zone 12 and is fed to a separator 22. The separator 22 may be a multi-stage fractionation unit, distillation system or similar known apparatus. The separator 22 forms a first fraction of paraffins 24, a second fraction of paraffins 26, and a third fraction of paraffins 28. In the exemplary embodiment, the separator 22 is a fractionation column, the first fraction of paraffins 24 is formed as an overhead stream, i.e., it exits at or near the top of the column, the second fraction of paraffins 26 is formed as a sidedraw fraction, i.e., it exits from the side of the column between the top and bottom, and the third fraction of paraffins 28 is formed as a bottom stream, i.e., it exits at or near the bottom of the column.

In an exemplary embodiment, the first fraction of paraffins 24 comprises paraffins with carbon chain lengths of C₄ to C₉, the second fraction of paraffins 26 comprises paraffins with carbon chain lengths of C₁₀ to C₁₃, and the third fraction of paraffins 28 comprises paraffins with carbon chain lengths of C₁₄ to C₂₂. As described herein, streams or fractions indicated as including a subject range of components generally include at least about 50 weight % of the subject range of components. In other embodiments, each fraction of paraffins 24, 26 and 28 has carbon chain lengths having a lower limit of C_{L}, where L is an integer from four (4) to thirty-one (31), and an upper limit of Cᵤ, where U is an integer from five (5) to thirty-two (32). The second fraction of paraffins 26 may have carbon chains shorter than, longer than, or a combination of shorter and longer than, the chains of the first fraction of paraffins 24. Likewise, the third fraction of paraffins 28 may have carbon chains shorter than, longer than, or a combination of shorter and longer than, the chains of the first fraction of paraffins 24 or the second fraction of paraffins 26.

The first fraction of paraffins 24 is sent to a reforming zone 30. In the reforming zone 30, paraffins and naphthenes may be converted to one or more aromatic compounds to produce reformate 32. An exemplary reformate 32 includes C₆-C₈ hydrocarbons. The reforming process also forms a substantial amount of hydrogen that exits the reforming zone 30 and is fed to the deoxygenation zone as hydrogen stream 14. Typically, the reforming zone 30 runs at high severity in order to maximize the production of one or more aromatic compounds. This high severity operation also can remove nonaromatic hydrocarbons in the C₈₊ portion of reformate 32, and thus can eliminate the separation of nonaromatics from the aromatic C₈ and C₉.

In the reforming zone 30, the first fraction of paraffins 24 is contacted with a reforming catalyst under reforming conditions. The reforming catalyst may be composed of a first component of a platinum-group metal, a second component of a modifier metal, and a third component of an inorganic-oxide support, which can be high purity alumina. Generally, the platinum-group metal is about 0.01 to about 2.0%, by weight, and the modifier metal component is about 0.01 to about 5%, by weight based on the total weight of the reforming catalyst. The balance of the catalyst composition can be alumina to sum all components up to about 100%, by weight. The platinum-group metal can be platinum, palladium, rhodium, ruthenium, osmium, or iridium. An exemplary platinum-group metal component is platinum. The metal modifier may include rhenium, tin, germanium, lead, cobalt, nickel, indium, gallium, zinc, uranium, dysprosium, thallium, or a mixture thereof. Exemplary reforming conditions include a liquid hourly space velocity of about 0.5 to about 15.0 hr⁻¹, a ratio of hydrogen to hydrocarbon of about 0.5 to about 10 moles of hydrogen per mole of hydrocarbon feed entering the reforming zone 30, and a pressure of about 69 to about 4830 kPa (about 10 to about 700 psi).

The reformate 32 from the reforming zone 30 is fed to an aromatic extraction zone 34. The extraction zone 34 can produce a non-aromatic stream 36 and an aromatic stream 38 rich in aromatics, such as benzene, toluene and xylenes, that can be sent to a fractionation zone 40. The non-aromatic stream 36 may be converted into additional aromatics by recycling to the reforming zone 30. As shown, a purge stream 37 is provided to remove materials that are too light or that will not convert to aromatic rings. The extraction zone 34 can utilize an extraction process, such as extractive distillation, liquid-liquid extraction or a combined liquid-liquid extraction/extractive distillation process. In an exemplary embodiment, extractive distillation is utilized with a main distillation column and a recovery column.

Extractive distillation can separate components having nearly equal volatility and having nearly the same boiling point. Typically, a solvent is introduced into a main extractive-distillation column above the entry point of the hydrocarbon stream being extracted. The solvent may affect the volatility of the components of the hydrocarbon stream boiling at different temperatures to facilitate their separation. Exemplary solvents include tetrahydrothiophene 1,1-dioxide, i.e. sulfolane, n-formylmorpholine, i.e., NFM, n-methylpyrrolidone, i.e., NFP, diethylene glycol, triethylene glycol, tetraethylene glycol, methoxy triethylene glycol, or a mixture thereof. Other glycol ethers may also be suitable solvents alone or in combination with those listed above.

At the fractionation zone 40, the aromatic stream 38 is fractionated into a benzene stream 42 and heavier aromatics 44, i.e., C₇-C₈ aromatics. The heavier aromatics 44 are fed to a dealkylation unit 46, such as a thermal hydrodealkylation unit. In the dealkylation unit 46, alkyl chains are stripped from benzene ring containing compounds to form a recovered benzene-rich stream 48 that is returned to the fractionation zone 40. The stripped alkyl chains are removed in an overhead stream containing methane, ethane and propane. Heavier aromatics 52, such as C₈₊ aromatics, exit the dealkylation unit 46 as a bottom stream. An exemplary dealkylation unit 46 is a small consumer of hydrogen. Hydrogen may be fed to the dealkylation unit 46. For example a portion 96 of hydrogen or as hydrogen stream 99, as described below.

In FIG. 1, the third fraction of paraffins 28 formed by the separator 22 is fed to an isomerization/cracking zone 60. As shown, a portion 62 of the hydrogen stream 14 is fed to the isomerization/cracking zone 60. The isomerization/cracking zone 60 isomerizes and/or cracks components of the third fraction of paraffins 28 to create the isoparaffins of equal or lighter molecular weight than the second fraction of paraffins 26. The isomerization reactions may include isomerization of n-paraffins to isoparaffins, the isomerization of alkylcyclopentanes to alkylcyclohexanes, the isomerization of cyclohexanes to alkylcycloparaffins, the hydroisomerization of n-paraffins to isoparaffins, and the isomerization of substituted aromatics. Further, isomerization/cracking zone 60 facilitates hydrocracking of paraffins and olefins. As a result, a jet range fuel stream 64, a diesel range fuel stream 66, light components 68 such as C₅₋ components, and an isomerized/cracked stream 70 are formed and separated in the isomerization/cracking zone 60. An exemplary isomerized stream 70 includes mainly C₆-C₈ range isoparaffins and few normal paraffins. The isomerized stream 70 is fed to the reforming zone 30 for reforming with the first fraction of paraffins 24 and the non-aromatic stream 36.

The isomerization/cracking zone 60 may be operated at mild conditions for mild cracking. In an exemplary embodiment, the isomerization/cracking zone 60 is operated at a temperature of from about 150 to about 425° C., such as about 285 to about 400° C. In an exemplary embodiment, the isomerization/cracking zone 60 is operated at a pressure of about 0 to about 17000 kPa, such as about 2000 to about 8200 kPa. Exemplary isomerization catalysts contain a supported Group VM noble metal, e.g., platinum or palladium, one or more Group VIII base metals, e.g., nickel, cobalt, and/or a Group VI metal, e.g., molybdenum. The support for the metals can be any refractory oxide or zeolite or mixtures thereof. Exemplary supports include silica, alumina, silica-alumina. silica-alumina phosphates, titania, zirconia, vanadia and other Group III, IV, or VA or VI oxides, as well as Y sieves.

As shown, a unit 98 for recovering hydrogen may be positioned along stream 68. For example, the unit 98 may be a reforming unit, such as a steam reforming unit. As shown, a water stream 97 may be fed to such a reforming unit 98. Stream 68 may be reformed to form a reformed stream 69 and a recovered hydrogen stream 99. As shown, hydrogen stream 99 may be directed to the dealkylation unit 46 or recycled back to hydrogen stream 14.

The second fraction of paraffins 26 formed by the separator 22 is fed to a linear alkylbenzene production zone 80. The benzene stream 42 is also fed to the linear alkylbenzene production zone 80. In the linear alkylbenzene production zone 80, the normal paraffins are catalytically dehydrogenated into mono-olefins of the same carbon numbers as the normal paraffins. Di-olefins (i.e., dienes) and aromatics are also produced as a result of the dehydrogenation reactions as expressed in the following equations:

Mono-olefin formation: C_{X}H_{2X+2}→C_{X}H_{2X}+H₂

Di-olefin formation: C_{X}H_{2X}→C_{X}H_{2X-2}+H₂

Aromatic formation: C_{X}H_{2X-2}→C_{X}H_{2X-6}+2H₂

Diolefins and aromatics are typically removed from the primarily mono-olefin stream prior to feeding the mono-olefin stream to the alkylation reactor zone.

To produce linear alkylbenzenes, the mono-olefins and the benzene are alkylated using an alkylation catalyst, such as a solid acid catalyst, that supports alkylation of the benzene with the mono-olefins. Hydrogen fluoride (HF) and aluminum chloride (AlCl₃) are two catalysts in commercial use for the alkylation of benzene with linear mono-olefins and may be used in the linear alkylbenzene production zone 80. Additional catalysts include zeolite-based or fluoridate silica alumina-based solid bed alkylation catalysts (for example, FAU, MOR, UZM-8, Y, X RE exchanged Y, RE exchanged X, amorphous silica-alumina, and mixtures thereof, and others known in the art). As a result of alkylation, a linear alkylbenzene is formed according to the reaction:

C₆H₆+C_{X}H_{2X}→C₆H₅C_{X}H_{2X+1}

To optimize the alkylation process, surplus amounts of benzene in benzene stream 42 may be supplied to the alkylation unit. Therefore, in some embodiments, the alkylation effluent may include benzene. Other embodiments may include no excess benzene. Further the alkylation effluent may also include some unreacted paraffins. The linear alkylbenzene production zone 80 includes a separation unit, such as a fractionation column, for separating the components in the alkylation effluent, such as linear alkylbenzene, a stream of C₁₆₊ linear alkylbenzenes, a stream of C₁₀-C₁₃ aromatics, and a stream of C₉₋ components. As shown, a linear alkylbenzene product stream 82 is separated.

Other products from dehydrogenation and alkylation include a light component stream 84, such as C₅₋ components, a C₁₀-C₁₃ aromatics stream 86, a heavy alkylate stream 88, and a hydrogen stream 90. As shown, the hydrogen stream 90 is fed back to the deoxygenation zone 12 in hydrogen stream 14. A portion 62 of the hydrogen stream 90 may be fed to the isomerization/cracking zone 60.

As a result of the linear alkylbenzene production processing, the linear alkylbenzene product stream 82 is isolated and exits the apparatus 10. It is noted that such separation processes are not necessary in all embodiments in order to isolate the linear alkylbenzene product stream 82. For instance, the linear alkylbenzene product stream 82 may be desired to have a wide range of carbon chain lengths and not require any fractionation to eliminate carbon chains longer than desired, i.e., heavies, or carbon chains shorter than desired, i.e., lights. Further, the renewable oil 11 may be of sufficient quality that no fractionation is necessary despite the desired chain length range.

In certain embodiments, the renewable oil 11 is substantially homogeneous and comprises free fatty acids within a desired molecular weight range. For instance, the feed may be entirely lauric free fatty acid, such that all the free fatty acids of the feed have 12 carbons. Alternatively, the renewable oil 11 may comprise triglycerides and free fatty acids that all have carbon chain lengths appropriate for a desired alkylbenzene product. In certain embodiments, the renewable oil 11 comprises coconut oil, palm kernel oil, babasu oil, castor oil, engineered microbial oils, or vegetable or animal oils modified to have internal hydroxyl groups along their fatty acid chains such that they behave similar to castor oil upon deoxygenation.

In certain embodiments, the natural oil source is castor, and the feed 14 comprises castor oils. Castor oils consist essentially of C₁₈ fatty acids with an additional, internal hydroxyl groups at the carbon-12 position. For instance, the structure of a castor oil triglyceride is:

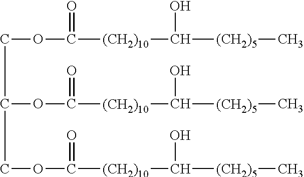

During deoxygenation of a feed 14 comprising castor oil, it has been found that some portion of the carbon chains are cleaved at the carbon-12 position. Thus, deoxygenation creates a group of lighter paraffins having C₁₀ to C₁₁ chains resulting from cleavage during deoxygenation, and a group of non-cleaved heavier paraffins having C₁₇ to C₁₈ chains. The lighter paraffins may form the first portion of paraffins 24 and the heavier paraffins may form the second portion of paraffins 26. It should be noted that while castor oil is shown as an example of oil with an additional internal hydroxyl group, others may exist. Also, it may be desirable to engineer genetically modified organisms to produce such oils by design. Another embodiment may involve a pathway such as found in U.S. patent application Ser. No. 13/712,181. As such, any oil with an internal hydroxyl group may be a desirable feed oil. Also, it may be desirable to modify regular vegetable oils, such as soybean or jatropha oil such that they contain internal hydroxyl groups, similar to the structure of castor oil, and thus the modified oil would behave similarly to castor oil upon deoxygenation, producing a first and second portion of paraffins. Examples of such modifications are described in U.S. patent application Ser. No. 13/712,181 hereby incorporated by reference in its entirety.

While hydrogen recovery unit 98 is illustrated as processing stream 68, additional or alternative hydrogen recovery units 98 may be used. For example, a hydrogen recovery unit 98 could be placed on streams 16, 37, 50, 52, 64, 66, 68, and/or 84, or others. In certain embodiments, sufficient hydrogen may be recovered from the hydrogen recovery units 98 such that the apparatus 10 need not receive any additional hydrogen, i.e., the apparatus 10 consumes no hydrogen. In embodiments in which the apparatus 10 consumes hydrogen, a hydrogen stream 94 may be fed to the hydrogen system within apparatus 10 for consumption in any of the hydrogen-consuming zones.

Embodiments of methods and apparatuses for producing a linear alkylbenzene product from renewable oils are described herein. The methods and apparatuses generate both benzenes and paraffins from the same renewable oil feed. Further, the methods and apparatuses recycle all hydrogen recovered during processing and recycle the hydrogen to deoxygenation or isomerization/cracking, or THDA zones, thus reducing or eliminating the introduction of fresh hydrogen.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the claimed subject matter in any way. Rather, the foregoing detailed description will provide those skilled in the art with a convenient road map for implementing an exemplary embodiment or embodiments. It being understood that various changes may be made in the function and arrangement of elements described in an exemplary embodiment without departing from the scope set forth in the appended claims.

What is claimed is:

1. A method for producing a linear alkylbenzene product from a renewable oil, the method comprising:
deoxygenating the renewable oil in a deoxygenation zone to form a paraffin stream;
fractionating the paraffin stream to form a first fraction of paraffins and a second fraction of paraffins;
processing the first fraction of paraffins in a reforming zone to form a reformate stream;
recovering a first hydrogen stream from the reforming zone;
forming a linear alkylbenzene stream in a linear alkylbenzene production zone from the second fraction of paraffins and a portion of the reformate stream;
recovering a second hydrogen stream in the linear alkylbenzene production zone; and
recycling the first hydrogen stream from the reforming zone and the second hydrogen stream from the linear alkylbenzene production zone, wherein substantially all hydrogen recovered from the reforming zone and the linear alkylbenzene production zone is recycled.

2. The method of claim 1 further comprising:
separating aromatics from non-aromatics in the reformate stream; and
fractionating the aromatics to form a benzene stream, wherein the portion of the reformate stream fed to the linear alkylbenzene production zone comprises the benzene stream.

3. The method of claim 2 wherein fractionating the aromatics further forms a C₇₊ aromatics stream and wherein the method further comprises dealkylating the C₇₊ aromatics stream to form additional benzene.

4. The method of claim 2 wherein forming the linear alkylbenzene stream in the linear alkylbenzene production zone comprises:
dehydrogenating the second fraction of paraffins to produce mono-olefins; and
reacting the benzene stream and the mono-olefins to produce the linear alkylbenzene product.

5. The method of claim 2 wherein fractionating the aromatics further comprises forming a fuel gas stream and a C₈₊ aromatics stream.

6. The method of claim 2 further comprising recycling the non-aromatics to the reforming zone.

7. The method of claim 1 wherein the linear alkylbenzene stream comprises C₁₀-C₁₆ linear alkylbenzenes, and wherein the method further comprises:
forming a stream of C₁₆₊ linear alkylbenzenes, a stream of C₁₀-C₁₃ aromatics, and a stream of C₉₋ components in the linear alkylbenzene production zone.

8. The method of claim 1 wherein fractionating the paraffin stream comprises forming the first fraction of paraffins, the second fraction of paraffins, and a third fraction of paraffins, and wherein the method further comprises:
isomerizing or cracking the third fraction of paraffins in an isomerization or cracking zone to form an isomerized/cracked stream; and
processing the isomerized/cracked stream with the first fraction of paraffins in the reforming zone.

9. The method of claim 8 further comprising feeding a portion of the first hydrogen stream and/or the second hydrogen stream to the isomerization or cracking zone.

10. The method of claim 9 wherein isomerizing or cracking the third fraction of paraffins in the isomerization zone forms a jet range fuel, a diesel range fuel, and C₅₋ hydrocarbons.

11. The method of claim 8 wherein fractionating the paraffin stream comprises forming the first fraction of paraffins comprising C₉₋ paraffins, forming the second fraction of paraffins comprising C₁₀-C₁₃ paraffins, and forming the third fraction of paraffins comprising C₁₄₊ paraffins.

12. The method of claim 11 wherein isomerizing or cracking the third fraction of paraffins comprises isomerizing or cracking the third fraction of paraffins to form the isomerized/cracked stream comprising C₆-C₈ paraffins.

13. The method of claim 1 wherein deoxygenating the renewable oil in the deoxygenation zone comprises deoxygenating palm kernel oil, coconut oil, babasu oil, castor oil, engineered microbial oils, or vegetable or animal oils modified to have internal hydroxyl groups along their fatty acid chains.

14. A method for producing a linear alkylbenzene product from a renewable oil, the method comprising:
deoxygenating the renewable oil to form a paraffin stream;
fractionating the paraffin stream to form an overhead fraction of paraffins, a sidedraw fraction of paraffins, and a bottom fraction of paraffins;
isomerizing or cracking the bottom fraction of paraffins to form an isomerized/cracked stream;
reforming the overhead fraction of paraffins and the isomerized/cracked stream to form a reformate stream and a first hydrogen stream; and
reacting the sidedraw fraction of paraffins with benzene from the reformate stream to form a linear alkylbenzene stream and a second hydrogen stream; wherein the renewable oil is deoxygenated in the presence of hydrogen from the first hydrogen stream and the second hydrogen stream to form the paraffin stream.

15. The method of claim 14 further comprising
separating aromatics from non-aromatics in the reformate stream; and
fractionating the aromatics to form a benzene stream, wherein the benzene from the reformate stream comprises the benzene stream.

16. The method of claim 15 wherein fractionating the aromatics forms the benzene stream and a C₇₊ aromatics stream, and wherein the method further comprises dealkylating the C₇₊ aromatics stream to form additional benzene.

17. The method of claim 15 wherein forming the linear alkylbenzene stream comprises:
dehydrogenating the sidedraw fraction of paraffins to produce mono-olefins; and
reacting the benzene stream and the mono-olefins to produce the linear alkylbenzene product.

18. The method of claim 14 further comprising adding a portion of the first hydrogen stream and/or the second hydrogen stream with the bottom fraction of paraffins for isomerizing/cracking the bottom fraction of paraffins.

19. The method of claim 18 wherein isomerizing/cracking the bottom fraction of paraffins forms a jet range fuel stream, a diesel range fuel stream, and a stream of C₅₋ hydrocarbons.

20. An apparatus for producing a linear alkylbenzene product from a renewable oil, wherein the apparatus comprises:
a deoxygenation zone for deoxygenating the renewable oil to form a paraffin stream;
a fractionation unit in fluid communication with the deoxygenation zone to receive and fractionate the paraffin stream into a first fraction of paraffins, a second fraction of paraffins, and a third fraction of paraffins;
a reforming zone in fluid communication with the fractionation unit to receive the first fraction of paraffins and to form a reformate stream and a first hydrogen stream, wherein the reforming zone recycles the first hydrogen stream within the apparatus;
a linear alkylbenzene production zone in fluid communication with the fractionation unit and with the reforming zone to receive the second fraction of paraffins and a portion of the reformate stream to form a linear alkylbenzene stream and second hydrogen stream, wherein the linear alkylbenzene production zone recycles the second hydrogen stream within the apparatus.
